(19) United States
(12) Patent Application Publication
(10) Pub. No.: US 2006/0270997 A1
(43) Pub. Date: Nov. 30, 2006

(54) Permission-based material dispenser

(75) Inventors: Bernard C.B. Lim, Oakville (CA); David G. Matsuura, Encinitas, CA (US); Philip J. Simpson, Escondido, CA (US); Mark Costa, Milton (CA); Hao Chen, Mississauga (CA); Kathleen Chancellor-Maddison, Hamilton (CA); Davis A.R. Kanbergs, Milton (CA)

Correspondence Address: BUCHANAN, INGERSOLL & ROONEY PC, POST OFFICE BOX 1404, ALEXANDRIA, VA, 22313-1404, US

(73) Assignee: VASOGEN IRELAND LIMITED, Shannon (IE)

(21) Appl. No.: 11/435,981

(22) Filed: May 18, 2006

Related U.S. Application Data
(60) Provisional application No. 60/683,280, filed on May 19, 2005.

(51) Int. Cl. A61M 5/00

(52) U.S. Cl. 604/181

(57) ABSTRACT

A syringe includes a releasable lock means for allowing discharge of a treated biological fluid sample to the patient in response to a release signal to the releasable lock means. The release signal is issued following a positive outcome from a verification process dependent upon temporal data from certain events in the collection, treatment and delivery of the biological fluid sample, and identity data of the patient and the syringe with the treated biological fluid.

### CROSS-REFERENCE TO RELATED APPLICATIONS

This application claims the benefit of priority to U.S. Provisional Application Ser. No. 60/683,280, filed May 19, 2005.

### BACKGROUND OF THE INVENTION

1. Field of the Invention

The present invention relates to the management of medical treatments. More specifically it relates to a permission-based fluid dispensing device.

2. Description of the Prior Art

Despite remarkable advances in health care technology and delivery, a large number of patients die or are disabled as a result of medical errors. These errors occur in health care settings, such as hospitals, clinics, nursing homes, urgent care centers, physicians' offices, pharmacies, and the care delivered in the home, and they usually result from systems problems rather than one single action or decision.

For many years, bar code labelling has been the technology of choice in ensuring patient safety. Recently, the Food and Drug Administration (FDA) issued a new rule which requires certain human drug and biological product labels to have bar codes. As such, the bar code for human drug products and biological products (other than blood, blood components, and devices regulated by the Center for Biologics Evaluation and Research) must contain the National Drug Code (NDC) number in a linear barcode. The rule is geared toward reducing the number of medication errors in hospitals and other health care settings by allowing health care professionals to use bar code scanning equipment to verify that the right drug (in the right dose and right route of administration) is being given to the right patient at the right time. The rule also requires the use of machine-readable information on blood and blood component container labels to help reduce medication errors.

However, bar codes require line of sight with a reader in order to be read and they cannot store additional information apart from simple identification data, such as a serial no. or a SKU. For example, a bar-coded wristband on a patient is not easy to read if the patient gets it wet or is sleeping on top of the arm bearing the wristband, or when the patient is on an emergency room gurney or operating table; these are instances where mistakes in medication or blood transfusion are most prevalent.

It is an object of the present invention to mitigate or obviate at least one of the above-mentioned disadvantages.

### SUMMARY OF THE INVENTION

In one of its aspects, the present invention provides a syringe for use with a patient in a biological fluid treatment system, the patient having a patient identifier, the syringe comprising:

a syringe inlet;

a syringe chamber for receiving the treated biological fluid;

a syringe outlet in communication with the chamber via a passage;

a syringe outlet valve to control the discharge of the treated biological fluid via the syringe outlet;

an incremental counter for recording temporal data corresponding to biological fluid treatment events, treated biological fluid events and delivery events;

a unique identifier associated with the syringe, the unique identifier correlatable to the patient identifier;

a releasable lock to operate the syringe outlet valve between a plurality of states;

a computer readable medium for storing the unique identifier, the patient identifier, temporal data, and data related to biological fluid treatment events, treated biological fluid events and delivery events;

a processor for comparing the unique identifier to the patient identifier to confirm the correlation between same; and for receiving the temporal data to determine at least one time delay between the events and for determining whether the at least one time delay is within a predefined range;

a release signal generator for issuing a release signal in response to an outcome from the processor, the release signal to operate the releasable lock.

In another aspect of the invention, there is provided a syringe device for use with a patient in a treatment process, the syringe device comprising:

a syringe inlet;

a syringe chamber for receiving the treated biological fluid;

a syringe outlet in communication with the chamber via a passage;

a syringe outlet valve to control the discharge of the treated biological fluid via the syringe outlet;

a releasable lock to operate the syringe outlet valve between a closed state, an open state and a permanently closed state;

an incremental counter for recording temporal data corresponding to biological fluid treatment events, treated biological fluid events and delivery events;

a unique identifier associated with the syringe, the unique identifier correlatable to the patient identifier;

a release signal generator for issuing a release signal to operate the releasable lock following acceptability of the temporal data and the correlation of the patient identifier and the unique identifier;

the releasable lock including:
a pivoted pawl member;
interconnected slots corresponding to the closed state, the open state and the permanently closed state;
   a first resilient member having a flange restricted to travel within the interconnected slots, wherein the first resilient means is spring made from a fuse material which temporarily changes consistency under the presence of the release signal, the position of flange within the interconnected slots dictating the state of the outlet valve.

Advantageously, the syringe can be irreversibly locked by placing the outlet valve in a permanent closed state, thus, subsequent use of the syringe is precluded, to substantially eliminate contamination risks.

### BRIEF DESCRIPTION OF THE DRAWINGS

These and other features of the preferred embodiments of the invention will become more apparent in the following detailed description in which reference is made to the appended drawings wherein:

FIG. 1 is a perspective view of a syringe;

FIG. 2 is a sectional view of the syringe of FIG. 1 taken along line 2-2′;

FIG. 3 is an exploded view of an outlet port of the syringe of FIG. 1;

FIG. 4 is a perspective view of an outlet valve;

FIG. 5 is a sectional view of the outlet valve element of FIG. 4 taken along line 5-5′;

FIG. 6(a) is a perspective view of the portion of locking mechanism in a locked state;

FIG. 6(b) is a perspective view of the a portion of locking mechanism in an open state;

FIG. 6(c) is a perspective view of the portion of locking mechanism in a permanently locked state;

FIG. 6(d) is a perspective view of the portion of locking mechanism adjacent to the outlet port of FIG. 3, in a permanently locked state;

FIG. 7 is a perspective view of the syringe with a needle coupled thereto and associated with a wristband;

FIG. 8 is a flowchart outlining the steps for a verification protocol; and

FIG. 9 is a flowchart outlining the steps for a verification portion.

### DESCRIPTION OF THE PREFERRED EMBODIMENTS

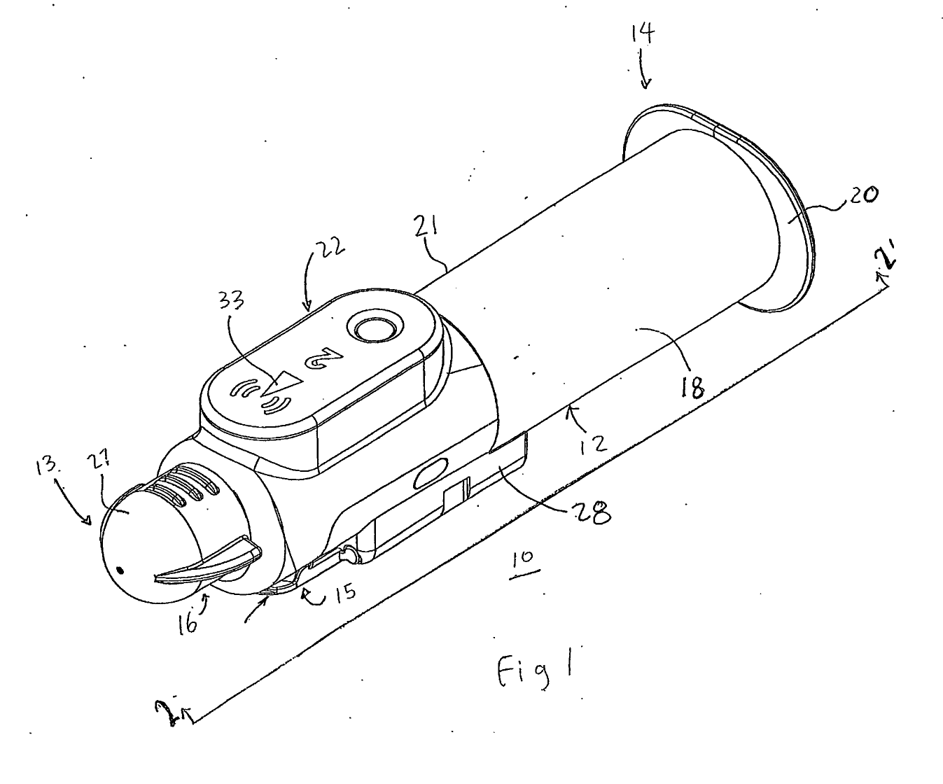

As shown FIG. 1, there is provided a syringe 10 for use in biological fluid treatment system to treat a biological fluid sample, such as a blood sample. Generally, the biological fluid treatment system includes a plurality of entities which are used at different stages during the handling of the blood sample, such as, a blood collection syringe to withdraw an untreated blood sample from a patient, a sample management unit, a blood treatment unit, a blood delivery syringe 10, and a patient identifier, such as wristband with an identification device. Following collection of the untreated blood sample, the blood collection syringe is coupled to the sample management unit with the blood delivery syringe 10 mounted thereon, and the sample management unit is introduced into the blood treatment unit, in which the untreated blood sample is subjected to one or more stressors, such as ozone or ozone/gas mixture, ultra-violet (UV) light and infra-red (IR) energy.

Following treatment, the treated blood sample is extracted to the blood delivery syringe 10, from which the treated blood sample is administered to the patient. At one or more critical stages, the system provides for a verification check, aimed at reducing the possibility of error, and thus ensure that the correct blood sample is returned to the correct originating patient. The verification check includes the steps of matching the blood sample, either in its treated or untreated form or both, with the originating patient. Typically, the wristband, the blood collection syringe, the blood delivery syringe 10, the sample management unit 12, may include circuitry for transmitting and receiving data related to the syringe and/or its contents, or a patient, such as identification data, SKU, serial no., manufacturing date, expiry date, fluid data, health facility data, health practitioner data, medication data, authentication data, and so forth. The data, or portions of the data, may also be secured via encryption algorithms and schemes, to ensure data integrity and/or authenticity of the entity. The circuitry may include, but is not limited to, a transmitter, a receiver, logic means or processor, a memory for data storage, a timing circuit, an antenna, a power source, input/out devices such as a display, an LED, a speaker, and a switch.

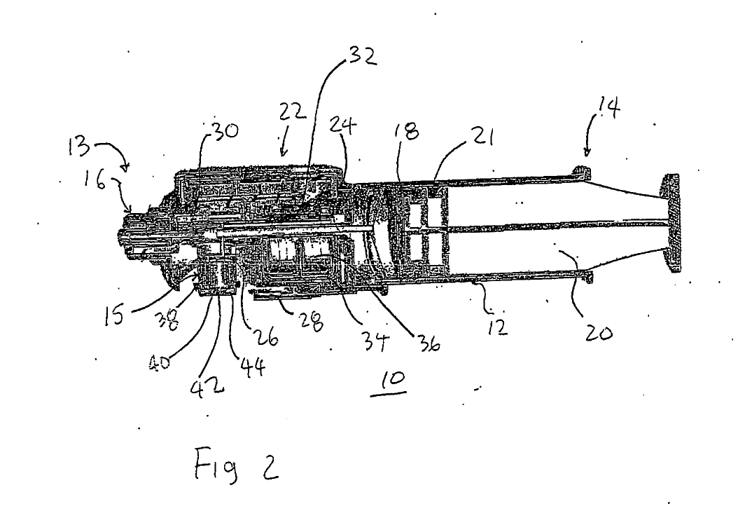

Below is a description of the post-treatment portion of the blood treatment process involving the use of the syringe 10 which ensures that the correct blood sample is returned to the correct originating patient. As shown in FIGS. 1 and 2, the syringe 10 includes a body portion 12 with a proximal end 13 and a distal end 14. Disposed at the proximal end 13 are an inlet port 15 and an outlet port 16. The syringe body portion 12 has a cylindrical cavity 18 which in cooperation with a plunger 20 provides a sample receiving chamber 21. The inlet port 15 is disposed at an angle to the outlet port 16, and intermediate the sample receiving chamber 21 and the outlet port 16. The plunger 20 is slidably disposed at the distal end 14 and is in tight fluid engagement with the cylindrical wall 18. The plunger 20 serves to draw fluid into the chamber 21 and urge the fluid therefrom. The syringe 10 also includes a channel portion 22 with a channel 24 in communication with the chamber 21 and the outlet port 16, and a channel 26 in communication with the inlet port 15 and the chamber 21 via a portion of the channel 24, as shown in FIG. 2. In order to prevent large particulate from entering the outlet port 16, an end cap 27 is removably attached thereto, while the inlet port 15 includes a slidable cap 28 to prevent contamination prior to use with the blood treatment unit 14. The treated blood sample is dispensed from the syringe 10 to the originating patient via the syringe outlet port 16 operable between an open position and a closed position by a releasable lock means 30, as will be described below.

Within the channel portion 22 is a printed circuit board (PCB) 32 having circuitry for transmitting, receiving and storing data related to the syringe and/or its contents or the originating patient. As described above, the circuitry includes, but is not limited to, a transmitter, a receiver, logic means or processor, a computer readable medium, a timing circuit, an antenna and a power source. Additionally, the circuitry includes RFID reader/writer functionality for reading RFID tags associated with entities within the treatment system. Also coupled to the PCB 32 are input/output devices such as a display, LED 33, a speaker or a button. In addition, the PCB 32 also includes circuitry for controlling the operation of the locking mechanism 30. A compartment 34 houses a power supply unit 36 comprising one or more batteries, and a power circuit resident on the PCB 32 for regulating the power therein and the input/output devices. The syringe 10 is typically maintained in a low power state, when not in use, to conserve battery energy. However, when the sample management unit is introduced into the blood treatment unit, the syringe 10 is placed into an operating state from the lower power state. Such a transition may be effected via a mechanical switch which is closed before insertion of the sample management unit into the blood treatment unit, or the switch is closed by the blood treatment unit following insertion of the sample management unit into the blood treatment unit. Other ways include an electronic switch actuable by an RF signal or a DC signal from the blood treatment unit, or a DC magnetic reed relay enabled by a magnet in the blood treatment unit. The batteries 36 may be removed after a single use of the syringe 10, in order to allow for proper recycling in compliance with environmental regulations. In order to facilitate easy battery installation or removal, the batteries 36 may be placed on a tray which is slidably received by the battery compartment 34.

As shown in FIG. 2, the syringe inlet port 15 includes bayonet pins 38 extending outwardly therefrom, which engage complementary grooves in a collar portion of a blood treatment chamber receptacle for coupling thereto. Similarly, a valve element 40 is located in the channel 26 and biased to a closed position against a valve seat 42 on an end cap 44 forming the outer end of the syringe inlet port 16. The valve element 40 is also aligned for abutment with a valve actuating element which is positioned in the chamber receptacle. The valve actuating element is thus operable to displace the valve element 40 from its closed position against the valve seat 42 to allow fluid flow therethrough.

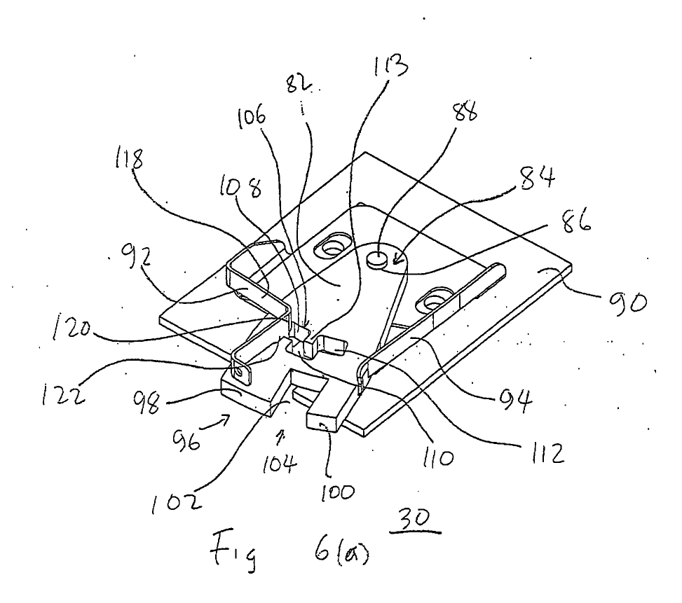

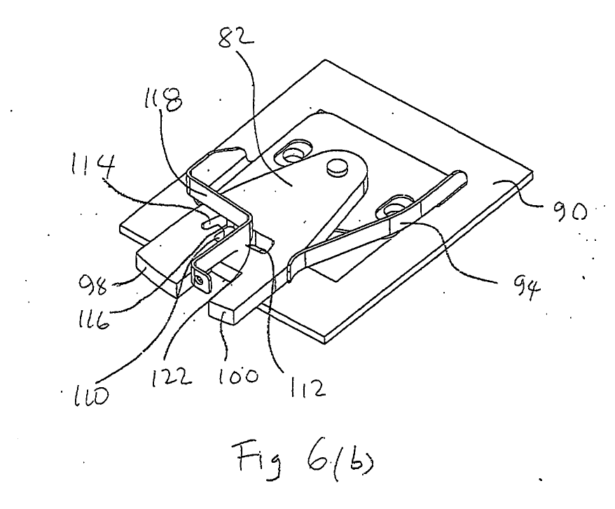

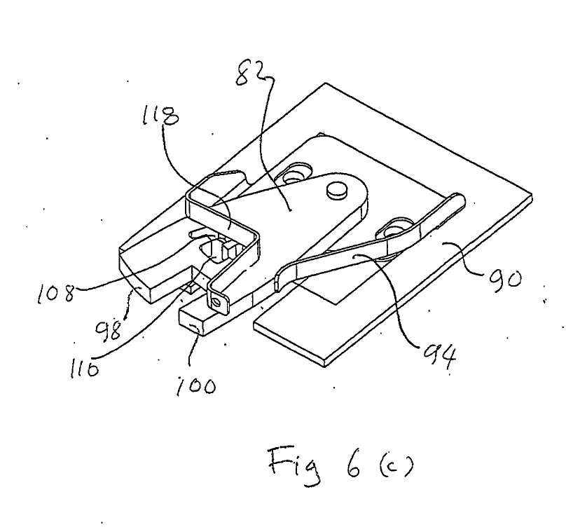

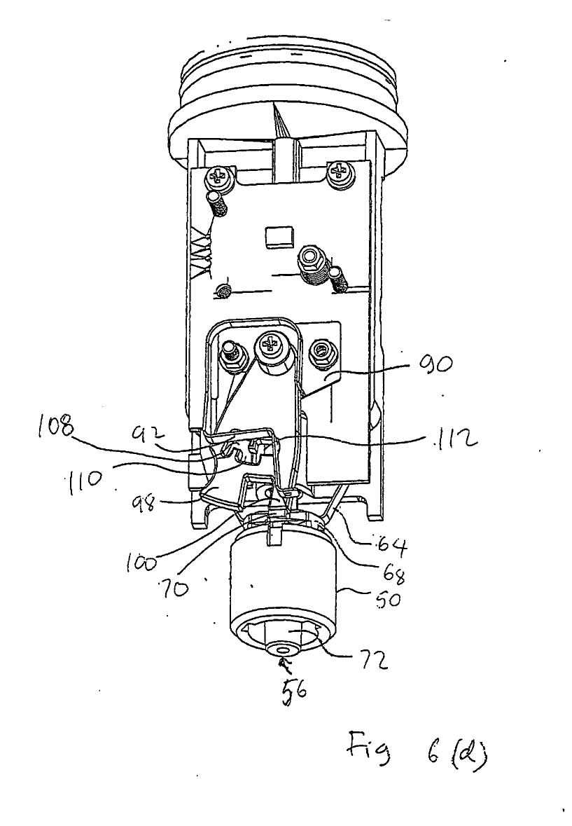

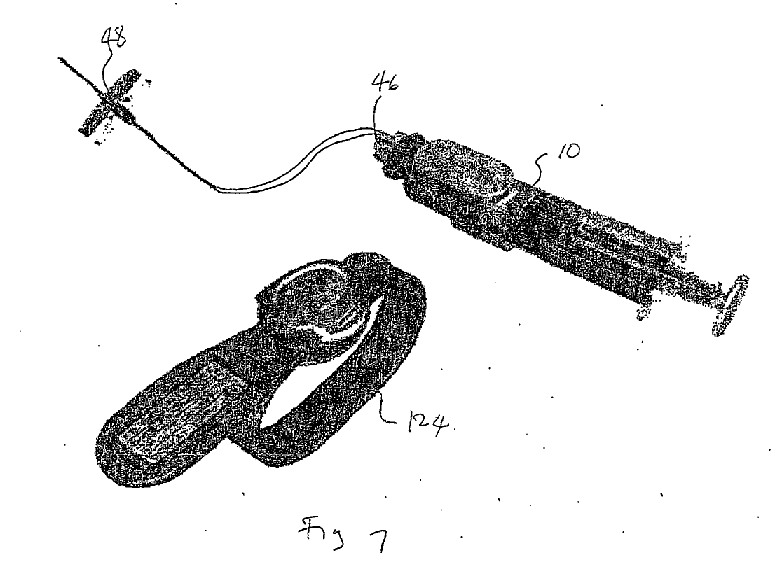

The blood transfer portion 22 is further provided with a releasable lock means shown generally at 30 for operating the syringe outlet port 16 between an open position and a closed position. As will be described, the locking mechanism 30 is operable in response to a release signal from the PCB 32, as shown in FIGS. 6(a) to 6(d). With the locking mechanism 30 unlocked, the syringe outlet port 16 is operable to form fluid coupling with a fluid fitting on a common blood sample delivery unit with a complementary Luer 46 or similar fitting, such as the needle 48, as shown in FIG. 7.

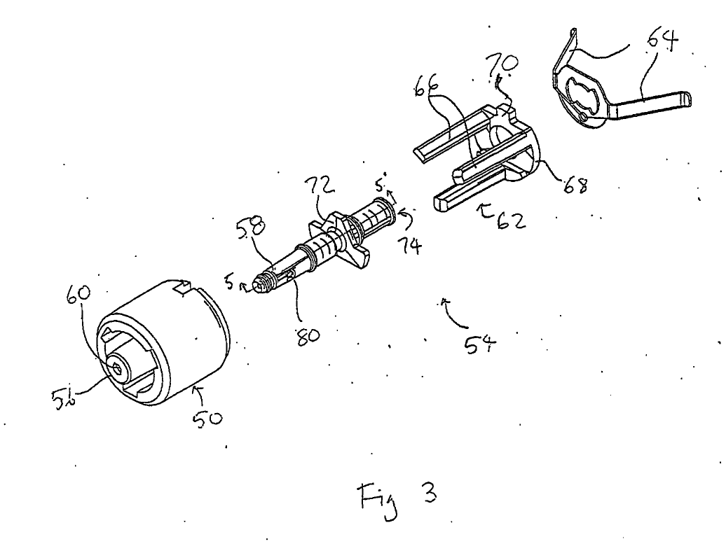

As best shown in FIG. 3, the syringe outlet port 16 includes a male Luer insert 50, an outlet valve means generally shown at 54 for opening and closing the access to the fluid channel 24 to control the flow of the blood sample therethrough. The male Luer insert 50 includes an opening 56 and a thread for the Luer fitting for coupling with female Luer 46 of a needle 48. The outlet valve means 54 includes a valve element portion 58, a valve seat portion 60, and first actuating means generally shown at 62 for actuating the valve element portion 58 relative to the valve seat portion 60. A pair of resilient members 64, such as a spring, biases the outlet valve means 54 in a closed position. As will be described, the first actuating means 62 is operable to displace the valve element portion 58 in different directions when the syringe body portion 20 is engaged or disengaged with a female Luer 46.

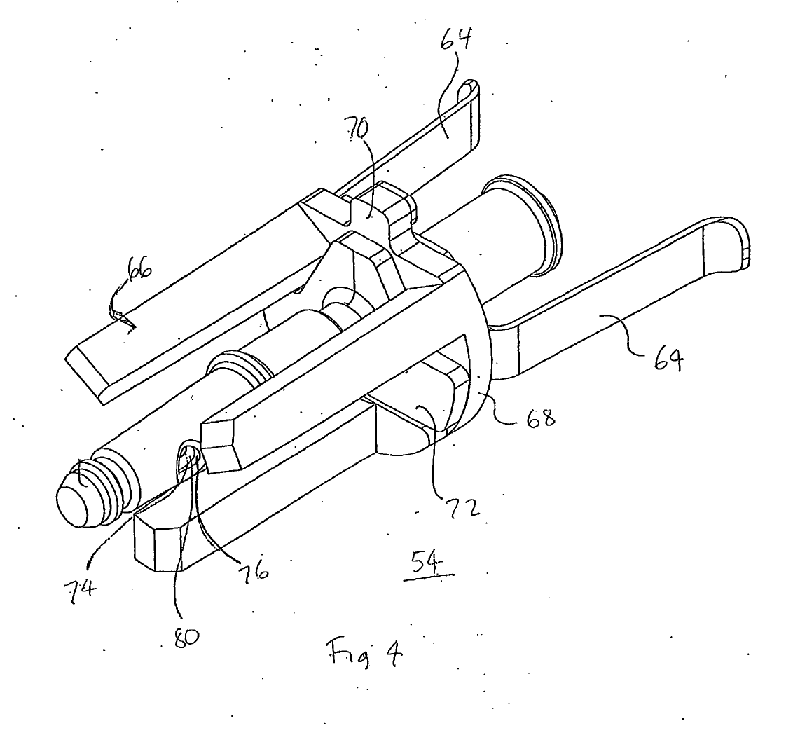

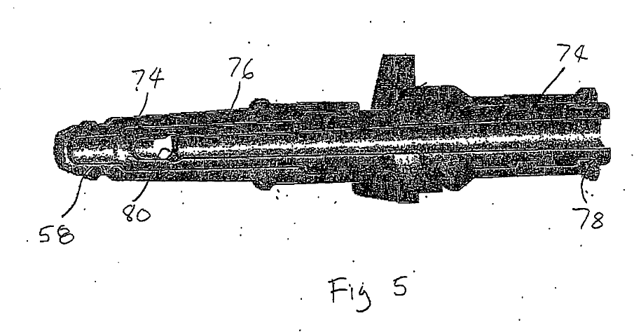

The first actuating means 62 takes the form of a plurality of first actuating elements 66 which extend outwardly from a central web 68, and also includes second actuating means such as a tab 70 extending therefrom. The central web 68 is fixed to a block 72 positioned in the channel 24 in the body portion 22 of the syringe 10, as shown in FIG. 2. The block 72 has a central bore 74 carrying a tubular valve stem 76 having one end carrying the valve element portion 58 and an opposite end carrying a valve stem head 78, which has a peripheral edge region with a sealing element such as an O-ring or the like, as shown in FIGS. 4 and 5. The valve stem 76 has a pair of fluid transfer holes, as shown at 80, immediately beside the valve element portion 58, thereby forming an inner valve passage in fluid communication with the chamber 21, as shown in FIGS. 4 and 5. The female Luer 46 includes complementary first actuating elements which displace the first actuating elements 66, when the female Luer 46 member is introduced into the male Luer insert 50. Subsequently, the first actuating elements 66 displace the valve stem 76 and the valve element portion 140 to open the central bore 74 within the valve stem 76 to the channel 26 to allow fluid flow through outlet port 16. The treated blood sample is dispensed from the syringe 10 to the originating patient via the syringe outlet port 16 operable between an open position and a closed position by a locking mechanism 30, as will now be described.

The outlet port 16 is operable between three states, a locked state, an open state and a permanently locked state, by a releasable lock means, such as locking mechanism 30, as shown in FIGS. 6(a) to 6(d). The locking mechanism 30 includes a pawl 82 coupled to the outlet valve means 54 to control the coupling of the female Luer 46 to the male Luer insert 50 of the syringe 10. The pawl 82 has one end 84 with an opening 86 for receiving a pivoting pin 88 protruding from a board 90 to allow pivoting thereabout. The pawl 82 is positioned between a first spring plate 92 and a second spring plate 94 which control its swinging motion. Typically, the first spring plate 92 is made from a fuse material or shape-memory wire (“muscle wire”), which temporarily changes consistency under the presence of a predetermined electric current signal, such as nickel titanium naval ordinance laboratory intermetallic material (NITINOL). Nitinol exhibits superelasticity and shape memory, such that nitinol is caused to heat up due to the predetermined electric current signal, as such it is mechanically deformed under stress above a specific temperature, and returns to the pre-stressed position when the stress is removed.

On the other end 96 of the pawl 82 is a first finger 98 and a second finger 100 defining a recess 102 with an opening 104. Adjacent to the recess 102 is a punched out slot 106 which includes a plurality of interconnected slots 108, 110, and 112. These interconnected slots 108, 110, and 112 correspond to the above-mentioned locked state, the open state and permanently locked state, respectively. The slots 108 and 112 are opposite each other and separated by a pawl tooth 113 on one side of the slot 106, and linked to one another by slot 110 on the other side of the slot 106. The slot 108 is L-shaped and includes one arm 114 and another arm 116 which links to slot 110.

The first spring plate 92 is secured to the board 90 at one end and includes an arcuate portion 118 positioned above the pawl 82. The arcuate portion 118 is bent at approximately 90 degrees at point 120, and adjacent thereto is an abutment flange 122 which engages the arm 114 of slot 108, in the locked position, as shown in FIG. 6(a). The subsequent positioning of the abutment flange 122 determines the operating state of the syringe 10.

The motion of the pawl 82 through the three different positions will now be described. Starting in the rest position, the abutment flange 122 is positioned in the arm 114 of slot 108. Upon receipt of the release signal following the verification process, a predetermined electric signal is caused to flow through the first spring plate 92, and the electric signal is sufficient to cause the first spring plate 92 to relax. The first spring plate 92 is sufficiently relaxed such that the second spring plate 94 forces the abutment flange 122 out of the arm 114 into arm 116, and finally into slot 110 corresponding to the open position, as shown in FIG. 6(b). A female Luer 46 of a needle 48 can now be attached to the syringe 10 and the treated blood is expressed from the chamber via the open outlet valve into the patient, as shown in FIG. 7.

After a predetermined time, such as 20 minutes, the predetermined electric signal is once again caused to flow through the first spring plate 92, and causes the first spring plate 92 to relax. The second spring plate 94 forces the abutment flange 122 out of the slot 110 into slot 112 corresponding to the permanently locked position, as shown in FIG. 6(c). If at the predetermined time, the female Luer 46 is still attached, the abutment flange 122 is not able to travel to the permanently locked position until the female Luer 46 is removed. By permanently locking the syringe 10, subsequent use of the syringe 10 is precluded, thus substantially eliminating contamination risks, as shown in FIG. 6(d).

The operation of the outlet valve means 54 in conjunction with the locking mechanism 30 will now be described with particular reference to FIGS. 6 to 9. In the locked position of the syringe 10, the tab 70 rests on the finger 98 and thus restricts the central web 68 from longitudinal displacement away from the opening 104. Any attempt to couple a female Luer 46 fails, since the complementary first actuating elements cannot displace the first actuating elements 66, and therefore the female Luer 46 and male Luer insert 50 cannot mate. Correspondingly, the outlet valve means 54 is biased closed by the pair of resilient members 64 acting on the central web 68, and thus the central bore 74 within the valve stem 76 is closed.

Upon energising the first spring plate 92, the pawl 82 is caused to rotate in a clockwise direction and the abutment flange 122 is forced out of the arm 114 into arm 116, and slides into slot 110 corresponding to the unlocked or open position. Concurrently, the finger 98 of the pawl 82 moves away from the tab 70 such that the tab 70 is now aligned with the recess 102. The female Luer 46 can now be introduced into the male Luer insert 50. As such, the complementary first actuating elements abut the first actuating elements 66 and the force applied to mate the female Luer 46 to the male Luer insert 50 displaces the first actuating element 66 away from the opening 104. The central web 68 moves in sympathy, and the tab 70 enters the recess 102 via the opening 104 and travels the length of the recess 102. The force applied to couple the LUERs 46 and 50 is sufficient to compress the resilient members 64 and thus open the central bore 74 within the valve stem 76.

As the treated blood often includes bubbles of gases used during treatment, therefore, the syringe 10 includes a de-bubbling system or bubble removal mechanism to expel gas from syringe. Alternatively, a separate vent cap is attached to the proximal end 13 to interface with the LUER 50. The vent cap includes a hydrophobic gas permeable membrane to prevent blood from escaping. Generally, more air can be introduced into the chamber 21 to coalesce the existing bubbles, thus facilitating removal of otherwise small bubbles. Thus, the barrel 13 is transparent such that a user can inspect the treated blood sample to verify that gas bubbles have been removed, after which the treated blood sample is ready for administration to the originating patient.

After the treated blood has been administered to the patient, the female Luer 46 is uncoupled from the male Luer insert 50, as the needle 48 is removed. With the complementary first actuating elements removed from the male Luer insert 50, the resilient members 64 expand to push the central web 68 towards the opening 56 and the tab 70 travels out of the recess 102 and faces the recess opening 104. At the predetermined time, a predetermined electric signal is caused to flow through the first spring plate 92, and the abutment flange 122 is forced out of the slot 110 into slot 112. The tab 70 now abuts the finger 100, and thus any longitudinal displacement of the central web 68 from away from the opening 56 is precluded. With the abutment flange 122 unable to be forced to return to slot 110, the syringe 10 is now permanently locked, and so a female Luer 46 can not be subsequently coupled to the male Luer insert 50, as shown in FIG. 6(d). In addition, following the administration of the blood sample to the patient, the syringe 10 is irreversibly disabled by electro/magnetic means to prevent further reading/writing of data on the computer readable medium, or irreversibly disabling the antenna coupled to the transceiver portion of the circuitry.

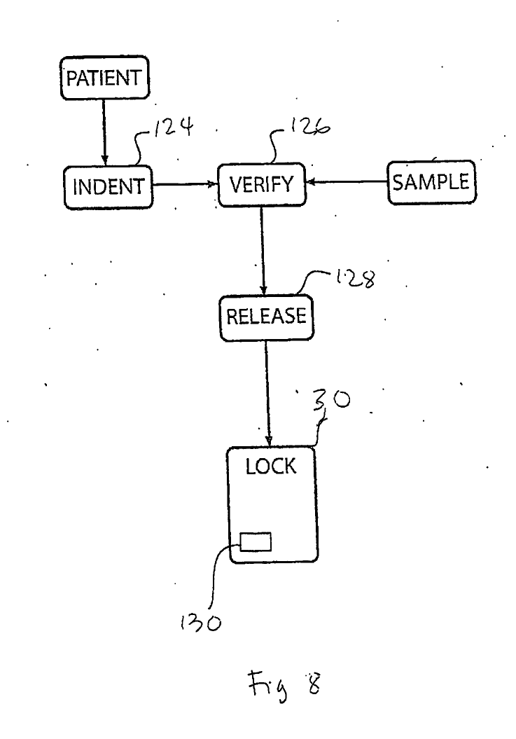

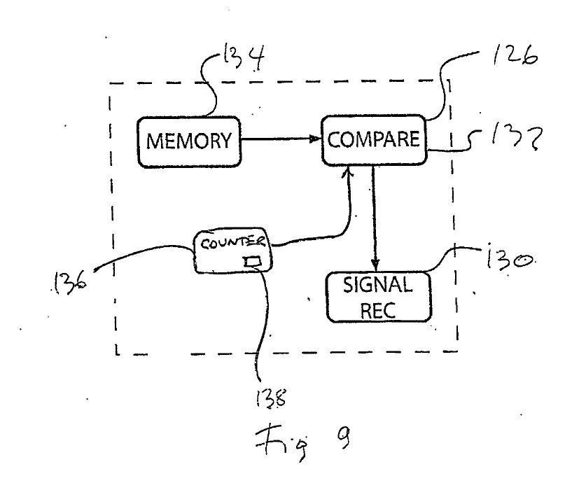

As will be described, the circuitry of PCB 32 of the syringe 10 includes portions responsible for performing a number of verification checks to ensure that the correct treated blood sample is delivered to the correct originating patient, and that certain events in the collection, treatment and delivery of the blood sample to the patient occur within prescribed time periods, as part of a verification. To that end, and as shown in FIGS. 8 and 9, the treatment system has identification means (Ident) 124 for identifying an originating patient, and the treated blood sample in the syringe 10Once the syringe 10 is in the operating state, the RFID reader/writer initiates polling for RFID tags within the vicinity, such as the wristband tag, to read the patient identity data on the wristband. A verification means 126 for verifies a match between the originating patient, and the treated blood sample in syringe 10, and release signal generating means 128 for generating a release signal in response to a positive verification by the verification means. The release signal is conveyed to the releasable locking mechanism 30 to deliver the predetermined current to the first spring plate 92, thereby to render the syringe 10 operable to deliver the treated blood sample to the originating patient. The releasable locking mechanism 30 has a signal receiving means 130 for receiving the release signal.

As shown in FIG. 9, the verification means 126 includes comparison means 132 for comparing patient identity data with treated blood sample identity data, both stored in memory means 134, and signal receiving means 130 to receive one or more signals associated with the originating patient identity data and/or the blood sample identity data. In this case, the one or more signals contain the originating patient identity data and/or the blood sample identity data. However, as an alternative, the one or more signals may contain data which is associated with or related to the patient or blood sample identity data. For example, the data in the signals may include one or more codes which allow the patient identity data or the blood sample identify data to be obtained from a data structure in the memory means 134 or some other location, for example in the form of a look-up table.

The verification means 126 also includes counter means 136 which provides temporal data related to a predetermined event including and/or between an untreated blood sample collection event and a treated blood sample delivery event. The temporal data may also include at least one elapsed time value between predetermined events related to an untreated blood sample collection event, a blood sample treatment event, or a treated blood sample delivery event. The counter means 136 may be implemented as an incremental counter 138 or a real-time clock. In this case, the incremental counter 138 tracks the events related to the treatment and post treatment events. The power supply 36 is sufficient to maintain substantial accuracy of the internal clock within the time period from collection of the untreated blood sample to the delivery of the treated blood sample to the patient. Therefore, the possibility of losing time or decreasing clock accuracy as the battery's power runs down is substantially eliminated.

Before treatment of the untreated blood sample, the verification means 126 is also operable to prevent treatment of the blood sample if the elapsed time value following the blood withdrawal from the patient has exceeded a predetermined value. Post-treatment, the verification means 126 issues an appropriate signal to the releasable locking means 30 to prevent opening of the syringe outlet 16 when the elapsed time value has exceeded a predetermined value. Also, the verification means 126 is operable to verify an identity match between the untreated blood sample in the syringe 10 and the originating patient, or a correlation between the identity data of same. Therefore, the syringe 10 then verifies whether the treated blood sample was withdrawn from originating patient, and a release signal is provided to the locking mechanism 30 to allow discharge of the blood.

The blood sample transfer portion 22 of the syringe 10 includes a filtered vent outlet (not shown) in the passage 62 for expelling one or more gas constituents in the treated blood sample. The vent outlet may also include a barrier layer which allows gaseous constituents in the blood sample to be expressed from the syringe 10 while retaining the treated blood sample therein

In another embodiment the circuitry may include a radio identification (RFID) integrated circuit associated with an antenna or an RFID tag. Additionally, any of the other above mentioned entities may include an RFID reader/writer associated with the afore-mentioned verification. As such, these other entities read the RFID tag on the syringe 10 or receive data from the computer readable medium to perform the verification check. The RFID tag on the syringe 10 is read by an RFID reader/writer, such the blood treatment unit 14 RFID reader/writer to verify authenticity of the syringe 10. Also, subsequent to the administration of the treated blood sample to the patient, the RFID tag on the syringe 10 receives a disable code from the blood treatment unit 14, thereby preventing the reuse of the syringe 10. Alternatively, the RFID tag may be rendered inoperable by an external signal causing a fuse to be blown therein or to destroy the antenna or receiver/transmitter.

In yet another embodiment, the system includes a locking mechanism 30 operable by a solenoid or motorized means configured to receive the release signal.

In another embodiment, the identification means, verification means and/or the release signal generating means may be located on other entities of the system 10. For example, verification means and/or the release signal generating means may be located on the wristband, or on the blood sample transfer portion 22, or the blood treatment unit.

The invention may be used with other autologous samples other than blood samples, such as bone marrow or, lymphatic fluids, semen, ova-fluid mixtures, other bodily fluids or other medical fluids which may or may not be “autologous”, for example fluid mixtures perhaps containing a patient desired solid sample such as from organs, body cells and cell tissue, skin cells and skin samples, spinal cords. The syringe 10 may also be used for medical testing where it is important to ensure that test results of a particular test can be delivered to the originating patient.

While the present invention has been described for what are presently considered the preferred embodiments, the invention is not so limited. To the contrary, the invention is intended to cover various modifications and equivalent arrangements included within the spirit and scope of the appended claims. The scope of the following claims is to be accorded the broadest interpretation so as to encompass all such modifications and equivalent structures and functions.

The embodiments of the invention in which an exclusive property or privilege is claimed are defined as follows:

1. A syringe for use with a patient in a biological fluid treatment system, the patient having a patient identifier, the syringe comprising:
a syringe inlet;
a syringe chamber for receiving the treated biological fluid;
a syringe outlet in communication with the chamber via a passage;
a syringe outlet valve to control the discharge of the treated biological fluid via the syringe outlet;
an incremental counter for recording temporal data corresponding to biological fluid treatment events, treated biological fluid events and delivery events;
a unique identifier associated with the syringe, the unique identifier correlatable to the patient identifier;
a releasable lock to operate the syringe outlet valve between a plurality of states;
a computer readable medium for storing the unique identifier, the patient identifier, temporal data, and data related to biological fluid treatment events, treated biological fluid events and delivery events;
a processor for comparing the unique identifier to the patient identifier to confirm the correlation between same; and for receiving the temporal data to determine at least one time delay between the events and for determining whether the at least one time delay is within a predefined range;
a release signal generator for issuing a release signal in response to an outcome from the processor, the release signal to operate the releasable lock.

2. The syringe of claim 1 wherein the syringe inlet is operable to form a first fluid coupling with a biological fluid treatment chamber outlet.

3. The syringe of claim 2 wherein the syringe outlet is operable to form a second fluid coupling with a medical accessory.

4. The syringe of claim 1 wherein the releasable lock being operable in response to a release signal to operate the syringe outlet valve between an open state and a closed state.

5. The syringe of claim 1 wherein the releasable lock is opened upon positive confirmation of the correlation between the patient identifier and the unique identifier, and provided that the at least one time delay is within a predefined range.

6. The syringe of claim 4 wherein the releasable lock is operable to place the outlet valve in an irreversible closed state.

7. The syringe of claim 2 wherein the syringe outlet includes a coupler engageable with a complementary coupler included with the medical accessory.

8. The syringe of claim 7 wherein the releasable lock includes a pivoted pawl member having a limited range of motion, interconnected slots corresponding to the locked state, the open state and an irreversible locked state, a first resilient member having a flange engaging the interconnected slots, and a second resilient member in cooperation with the first resilient member to control the range of motion, wherein the flange is restricted to travel within the interconnected slots.

9. The syringe of claim 8 wherein the first resilient member is spring made from a fuse material which temporarily changes consistency under the presence of the release signal.

10. The syringe of claim 9 wherein the fuse material is nickel titanium naval ordinance laboratory intermetallic material (NITINOL).

11. The syringe of claim 10 wherein the second resilient member forces the flange into a slot corresponding to the irreversible closed state of the outlet valve.

12. The syringe of claim 11 wherein the complementary coupler and the coupler can only form second coupling with the releasable lock in an open position.

13. The syringe of claim 12 wherein the outlet valve comprises a valve element portion and a valve seat portion, and an actuator for translating the valve element portion relative to the valve seat portion to open the syringe outlet valve, the actuator linked to the coupler and being actuable upon engaging the coupler with the complementary coupler.

14. A syringe for use with a patient in a biological fluid treatment system, the patient having a patient identifier, the syringe comprising:
a syringe inlet;
a syringe chamber for receiving the treated biological fluid;
a syringe outlet in communication with the chamber via a passage;
a syringe outlet valve to control the discharge of the treated biological fluid via the syringe outlet;
a releasable lock to operate the syringe outlet valve between a closed state, an open state and a permanently closed state;
an incremental counter for recording temporal data corresponding to biological fluid treatment events, treated biological fluid events and delivery events;
a unique identifier associated with the syringe, the unique identifier correlatable to the patient identifier;
a release signal generator for issuing a release signal to operate the releasable lock following acceptability of the temporal data and the correlation of the patient identifier and the unique identifier;
the releasable lock including:
a pivoted pawl member;
interconnected slots corresponding to the closed state, the open state and the permanently closed state;
a first resilient member having a flange restricted to travel within the interconnected slots, wherein the first resilient member is spring made from a fuse material which temporarily changes consistency under the presence of the release signal, the position of flange within the interconnected slots dictating the state of the outlet valve.

15. The syringe of claim 14 wherein the fuse material is nickel titanium naval ordinance laboratory intermetallic material (NITINOL).

16. The syringe of claim 15 wherein the releasable lock includes a second resilient member to force the flange into a slot corresponding to a permanently closed state.

17. The syringe of claim 16 including a channel portion having electronic circuitry for transmitting, receiving and storing data related to the syringe and/or its contents or the patient; the circuitry comprising a transmitter, a receiver, an antenna, processor, computer readable medium, a timing circuit for maintaining temporal data related to the treatment process, a power source and input/output devices.

18. The syringe of claim 16 wherein the electronic circuitry includes an RFID tag.

19. The syringe of claim 18 wherein the RFID tag is active, semi-active or passive.

20. The syringe of claim 17 wherein outlet valve comprises a filter in the passage for expelling one or more gas constituents in the treated sample.
